SEQUENCE LISTING (23 sequences)

SEQ ID NO: 1 - protein, 23 aa, Octopus mimus
GWLIRGAIHAGKAIHGLIHRRRH

SEQ ID NO: 2 - DNA, 20 bp, synthetic construct
  source 1..20: mol_type = other DNA
agcatgctttgagacaggac

SEQ ID NO: 3 - DNA, 22 bp, synthetic construct
  source 1..22: mol_type = other DNA
cacgaagagcgctacggaagtt

SEQ ID NO: 4 - DNA, 22 bp, synthetic construct
  source 1..22: mol_type = other DNA
gttcgtctcaaagttgctcctc

SEQ ID NO: 5 - DNA, 22 bp, synthetic construct
  source 1..22: mol_type = other DNA
gttcgtctcaaagttgctcctc

SEQ ID NO: 6 - DNA, 24 bp, synthetic construct
  source 1..24: mol_type = other DNA
caacaacatcatgacccgctacct

SEQ ID NO: 7 - DNA, 24 bp, synthetic construct
  source 1..24: mol_type = other DNA
gttctctgcctccgttctgtcctt

SEQ ID NO: 8 - DNA, 23 bp, synthetic construct
  source 1..23: mol_type = other DNA
ggagaggaggtctatctgctatg

SEQ ID NO: 9 - DNA, 20 bp, synthetic construct
  source 1..20: mol_type = other DNA
ggcttctggaggttctggtc

SEQ ID NO: 10 - DNA, 24 bp, synthetic construct
  source 1..24: mol_type = other DNA
tggcatgggagaatcagttacaag

SEQ ID NO: 11 - DNA, 19 bp, synthetic construct
  source 1..19: mol_type = other DNA
tgccccagccatctcatcc

SEQ ID NO: 12 - DNA, 22 bp, synthetic construct
  source 1..22: mol_type = other DNA
ccctcctagccctcactgtaaa

SEQ ID NO: 13 - DNA, 22 bp, synthetic construct
  source 1..22: mol_type = other DNA
ggatcttctcaatgaccttctt

SEQ ID NO: 14 - DNA, 18 bp, synthetic construct
  source 1..18: mol_type = other DNA
acagcagttgcatgggtg

SEQ ID NO: 15 - DNA, 18 bp, synthetic construct
  source 1..18: mol_type = other DNA
tggtctcctggtctttcc

SEQ ID NO: 16 - DNA, 21 bp, synthetic construct
  source 1..21: mol_type = other DNA
cgagcagtacgcgtgggagtc

SEQ ID NO: 17 - DNA, 24 bp, synthetic construct
  source 1..24: mol_type = other DNA
agcgtgatggggtagccgatgaac

SEQ ID NO: 18 - DNA, 18 bp, synthetic construct
  source 1..18: mol_type = other DNA
ccgtgctgtctggaggta

SEQ ID NO: 19 - DNA, 22 bp, synthetic construct
  source 1..22: mol_type = other DNA
aaggagcaagggaggtgatttc

SEQ ID NO: 20 - DNA, 22 bp, synthetic construct
  source 1..22: mol_type = other DNA
atgacaagcgcactcagagaga

SEQ ID NO: 21 - DNA, 23 bp, synthetic construct
  source 1..23: mol_type = other DNA
tcagtggaatgagtcggagtctc

SEQ ID NO: 22 - DNA, 22 bp, synthetic construct
  source 1..22: mol_type = other DNA
gcttgcagaaacgatcgtaaag

SEQ ID NO: 23 - DNA, 21 bp, synthetic construct
  source 1..21: mol_type = other DNA
ggccatcttgtccacatcata

(19) United States
(12) Patent Application Publication
(10) Pub. No.: US 2025/0041382 A1
(43) Pub. Date: Feb. 6, 2025

(54) ANTIVIRAL COMPOSITION COMPRISING OCTOMININ

(71) Applicant: NATIONAL MARINE BIODIVERSITY INSTITUTE OF KOREA, Chungcheongnam-Do (KR)

(72) Inventors: Ilson Whang, Chungcheongnam-Do (KR); Grace Choi, Chungcheongnam-Do (KR); Moongeun Yoon, Chungcheongnam-Do (KR)

(21) Appl. No.: 18/786,152

(22) Filed: Jul. 26, 2024

(30) Foreign Application Priority Data
Jul. 27, 2023 (KR) 10-2023-0098472

(51) Int. Cl. A61K 38/17; A61K 9/00; A61P 31/14

(52) U.S. Cl. CPC A61K 38/1767; A61K 9/0056; A61P 31/14

(57) ABSTRACT

The present disclosure relates to an antiviral composition comprising Octominin, an antibacterial peptide derived from Octopus minor, and can be useful as an antiviral agent against fish viruses by confirming that the Octominin exhibits antiviral activity against viral hemorrhagic sepsis virus (VHSV), infectious hematopoietic necrosis virus (IHNV), and infectious pancreatic necrosis virus (IPNV), which are viruses related to fish farming.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “70576_Seq-Listing.xml”, which was created on Jul. 22, 2024 and is 21,103 bytes in size. The subject matter of the Sequence Listing is incorporated herein in its entirety by reference.

### CROSS-REFERENCE TO RELATED APPLICATIONS

This application is based on and claims priority from Korean Patent Application No. 10-2023-0098472, filed on Jul. 27, 2023 with the Korean Intellectual Property Office, the disclosure of which is incorporated herein in its entirety by reference.

### TECHNICAL FIELD

The present disclosure relates to an antiviral composition comprising Octominin, an antibacterial peptide derived from Octopus minor, as an active ingredient.

### BACKGROUND

With the development of the aquaculture industry and the subdivision of aquaculture species, the spread of viral diseases is becoming a major problem in intensive aquaculture, and these viral diseases are spreading at an increasing pace, and new types of diseases are emerging every year. Viral infections cause mass mortality in aquaculture organisms and weaken the resistance of fish to lead to economic losses.

Fish disease viruses include Viral Hemorrhagic Septicemia Virus (VHSV), Infectious Hematopoietic Necrosis Virus (IHNV), Infectious Pancreatic Necrosis Virus (IPNV), etc., which are the main causes of the incidence of diseases in farmed fish to infect fish and cause mass mortality.

In particular, the VHSV has been mainly distributed in Europe, but recently, viral hemorrhagic sepsis has been detected in wild marine fish species in Japan and Korea and reported to cause fatal diseases in farmed halibut during low-temperature periods. The VHSV causes economic losses by causing deaths not only of small fish but also of large fish at the time of shipment.

In addition, the IHNV is known as a viral disease that causes mass mortality in salmonid fish worldwide. The disease causes up to 80% loss of fish in aquaculture farms, depending on fish species, fish sizes, virus strains, and environmental conditions.

In addition, the IPNV is also a virus that causes diseases in salmonid fish, mainly river trout and rainbow trout, and to date, it has been confirmed that more than 80 species of aquatic animals are infected with IPNV. Accordingly, the IPNV is a virus that causes infection in various fish species, and a very dangerous pathogen that results in a 100% mortality rate in severe cases when infecting river trout and rainbow trout.

The use of antiviral drugs also proposes new threats in the food industry, particularly aquaculture industry, due to significant risks such as residues in farmed fish. Antimicrobial peptides (AMPs) are promising as antiviral therapeutics in that there is little or no harm to the development of resistance and residual effects because the AMPS primarily affect the viral replication cycle by targeting specific mechanisms.

As a prior art, Korean Patent Publication No. 10-2020-0047329 discloses that Octominin or a fragment thereof has excellent antibacterial activity against microorganisms such as Candida, etc. In addition, Korean Patent Publication No. 10-2021-0043939 discloses that Octominin or a fragment thereof exhibits a high killing effect against Streptococcus parauberis without affecting antibiotic resistance.

Accordingly, the present inventors confirmed that Octominin, an antibacterial peptide derived from Octopus minor, exhibited antiviral activity against VHSV, IHNV, and IPNV while conducting research on an antiviral agent using an antimicrobial peptide, and then completed the present disclosure.

### SUMMARY

The present disclosure has been made to provide an antiviral composition for fish viral diseases.

The present disclosure has also been made to provide a composition for a fish feed additive having antiviral activity against fish viral diseases.

The present disclosure has also been made to provide a pharmaceutical composition for preventing or treating fish viral diseases.

The present disclosure has also been made to provide a method for preventing or treating fish viral diseases.

An embodiment of the present disclosure provides an antiviral composition for fish viral diseases including a peptide having an amino acid sequence as set forth in SEQ ID NO: 1 as an active ingredient.

Another embodiment of the present disclosure provides a composition for a fish feed additive having antiviral activity against fish viral diseases including the peptide as an active ingredient.

Yet another embodiment of the present disclosure provides a pharmaceutical composition for preventing or treating fish viral diseases including the peptide as an active ingredient.

Still another embodiment of the present disclosure provides a method for preventing or treating fish viral diseases including administering the peptide to fish.

According to the embodiments of the present disclosure, the present disclosure relates to an antiviral composition including Octominin, an antibacterial peptide derived from Octopus minor, and can be useful as an antiviral agent against fish viruses by confirming that the Octominin exhibits antiviral activity against viral hemorrhagic sepsis virus (VHSV), infectious hematopoietic necrosis virus (IHNV), and infectious pancreatic necrosis virus (IPNV), which are viruses related to fish farming.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a microscopic photograph of cytopathic activity of Octominin against fish pathogenic viruses VHSV, IHNV, and IPNV.

FIG. 2A shows a design of Octominin treatment experiment for a plaque-forming reduction assay.

FIG. 2B is a graph showing a virus plague reduction (%) to hours post infection (hpi) after virus challenge according to the experiment of FIG. 2A.

FIGS. 3A to 3H are graphs showing mRNA expression levels of immune response genes when treated with Octominin.

FIG. 3I is a heatmap for gene transcription levels in FIGS. 3A to 3H.

FIGS. 4A to 4H are graphs showing mRNA expression levels of immune response genes during IHNV challenge after pre-treatment with Octominin.

FIG. 4I is a heatmap for gene transcription levels in FIGS. 4A to 4H.

FIG. 5 is a graph showing the virus copy number of IHNV after IHNV challenge after pre-treatment with Octominin.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in detail.

The present disclosure provides an antiviral composition for fish viral diseases including a peptide having an amino acid sequence as set forth in SEQ ID NO: 1 as an active ingredient.

The peptide is a synthetic peptide (GWLIRGAIHAGKAIHGLIHRRRH, SEQ ID NO: 1) consisting of 23 amino acid residues named Octominin based on a protein derived from Octopus minor.

The fish viral disease may be a disease caused by at least one virus selected from the group consisting of viral hemorrhagic septicemia virus (VHSV), infectious hematopoietic necrosis virus (IHNV), and infectious pancreatic necrosis virus (IPNV).

The fish of the present disclosure may be applied to all species of fish that may be a host for the virus, and may be at least one species selected from the group consisting of halibut, rockfish, red sea bream, rock bream, sea bass, gray mullet, bass, Gizzard shad, flounder, puffer fish, mackerel, greenling, tuna, croaker, yellowtail, horse mackerel, salmon, rainbow trout, carp, leather carp, eel, catfish, loach, and crucian carp, but is not limited thereto.

Further, the present disclosure provides a composition for a fish feed additive having antiviral activity against fish viral diseases including the peptide as an active ingredient.

The composition for the fish feed additive according to the present disclosure may be prepared together with various ingredients commonly used. For example, the composition for the fish feed additive may include at least one protein source selected from the group consisting of marine proteins (e.g., fish meal or krill meal), vegetable proteins (e.g., soybean powder, wheat gluten, corn gluten, lupine powder, pea powder or sunflower seed powder), blood meal and bone meal, at least one energy source selected from the group consisting of fish oils (e.g., squid liver oil) or vegetable oils (e.g., rapeseed oil, bean oil, soybean oil), a mixture of vitamins and a mixture of minerals.

Further, the present disclosure provides a pharmaceutical composition for preventing or treating fish viral diseases including the peptide as an active ingredient.

In the present disclosure, the pharmaceutical composition may further include a pharmaceutically acceptable carrier.

In the present disclosure, the pharmaceutically acceptable carrier is generally used in preparation and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, mineral oils, etc., but is not limited thereto.

In the present disclosure, the pharmaceutical composition may further include a lubricant, a wetting agent, a sweetening agent, a flavoring agent, an emulsifier, a suspension, a preservative, and the like, in addition to the ingredients, but is not limited thereto.

In the present disclosure, the formulation of the pharmaceutical composition may be in the form of a solution in an oil or aqueous medium, a suspension or an emulsion, or in the form of extracts, powders, granules, tablets or capsules, but is not limited thereto.

In the present disclosure, the pharmaceutical composition may be administered orally or parenterally. In the case of parenteral administration, the pharmaceutical composition may be administered by intravenous injection, subcutaneous injection, intramuscular injection, intraperitoneal injection, transdermal administration, or the like. For example, the pharmaceutical composition may be administered orally.

In the present disclosure, a suitable dose of the pharmaceutical composition varies depending on factors, such as a formulation method, an administration method, age, weight, sex, and morbid condition of an animal, an administration time, an administration route, an excretion rate, and response sensitivity, and an ordinarily skilled physician may easily determine and prescribe a dose effective for desired treatment or prevention. For example, the composition of the present disclosure may be intraperitoneally injected or administered orally 1 to 5 times every 1 to 10 days at an effective ingredient content of 1 to 300 mg per 1 kg of a living body, but is not limited thereto.

Further, the present disclosure provides a method for preventing or treating fish viral diseases including administering the peptide to fish.

A specific dose, administration interval, etc. of the antiviral composition for viral diseases of the present disclosure may vary depending on farmed fish species, the farming density, a water temperature in the farm, etc. However, the antiviral composition may be applied to a method for preventing or treating fish viral diseases, characterized to be added to a fish farming tank at a concentration of 10 to 500 ppm for 1 to 7 days with reference to other farming additives.

In addition, the antiviral composition for fish may be mixed in feed and orally administered to be 1 to 500 mg/kg of fish body weight for a week or more to be applied to a method for preventing or treating fish viral diseases.

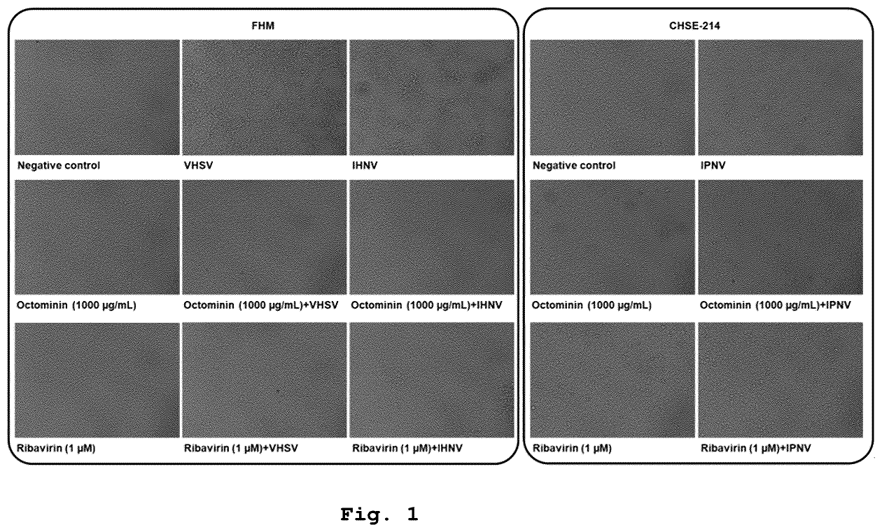

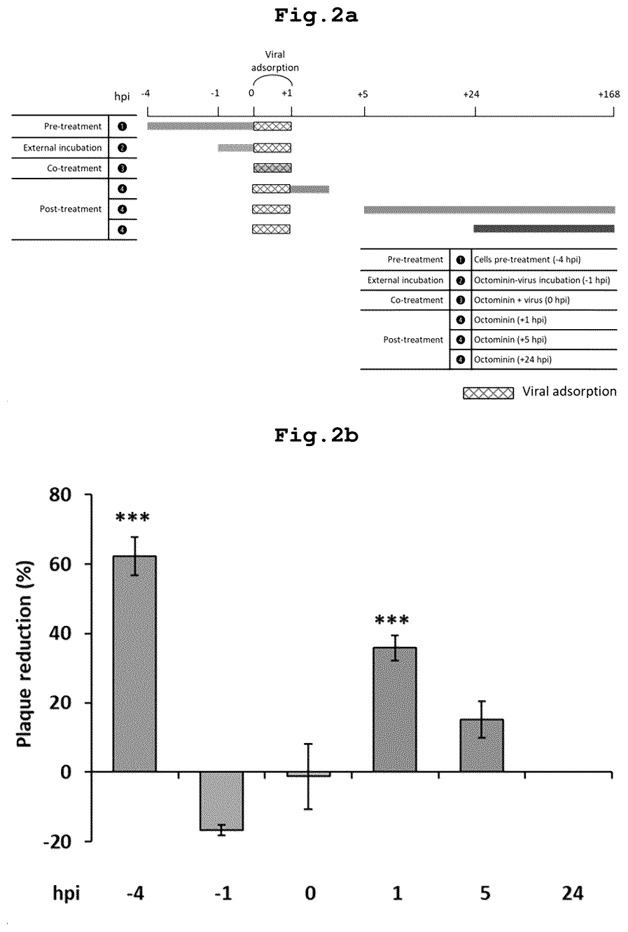

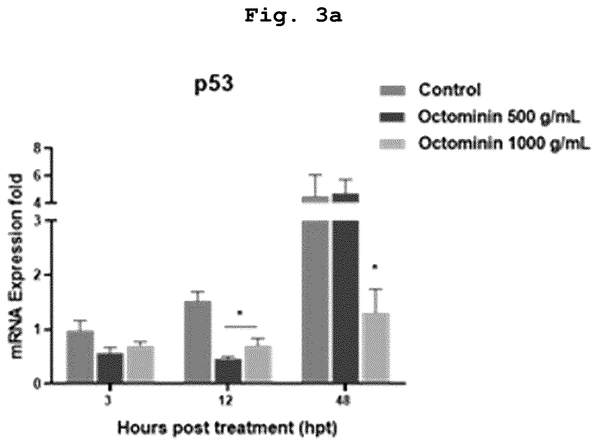

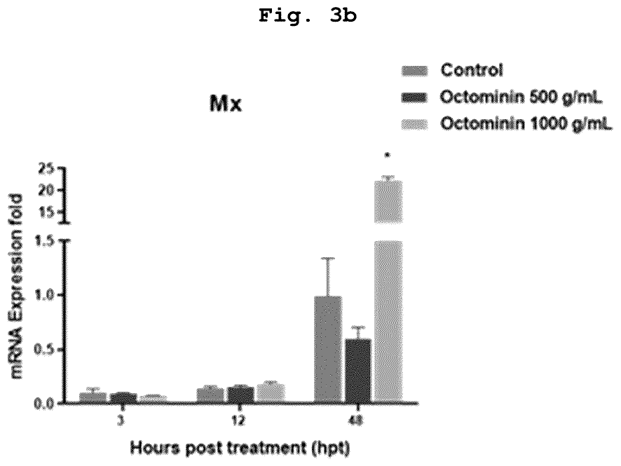

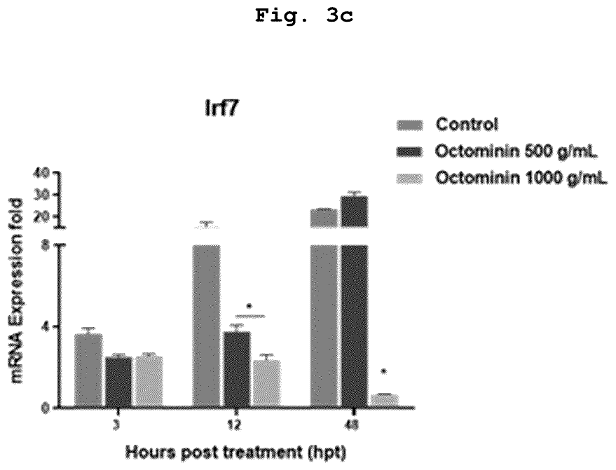

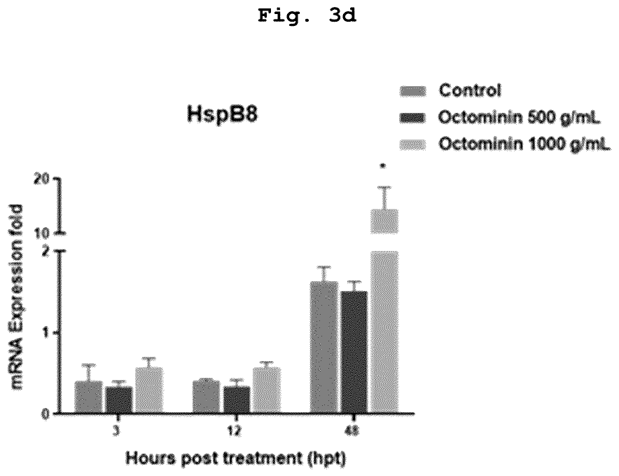

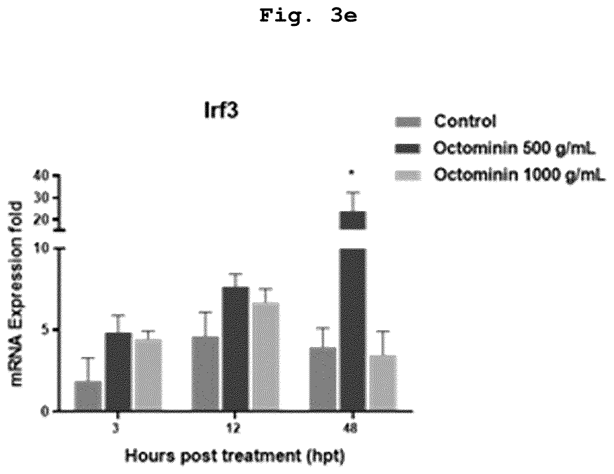

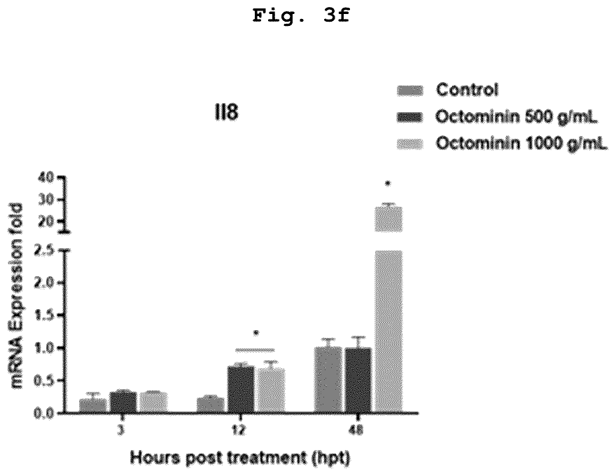

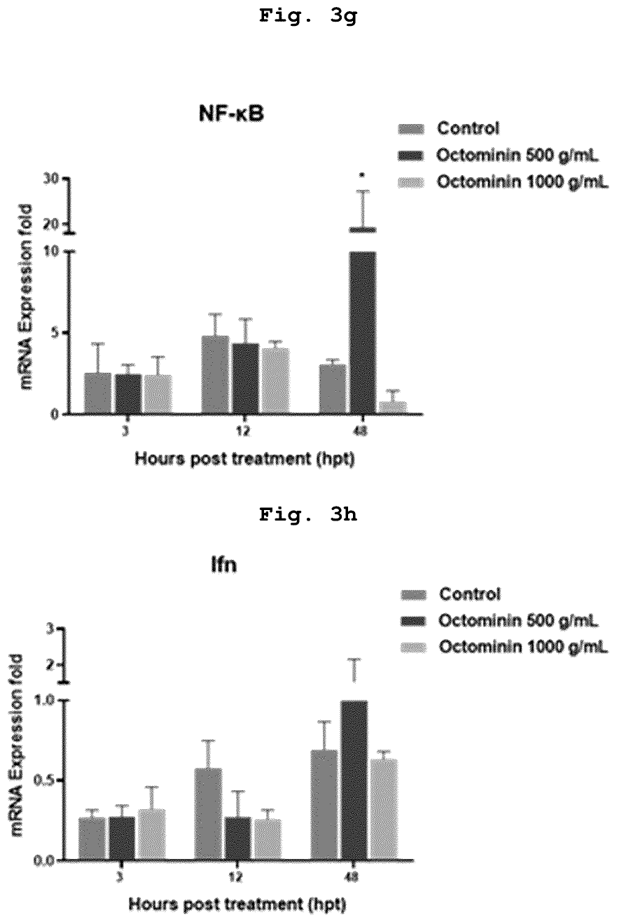

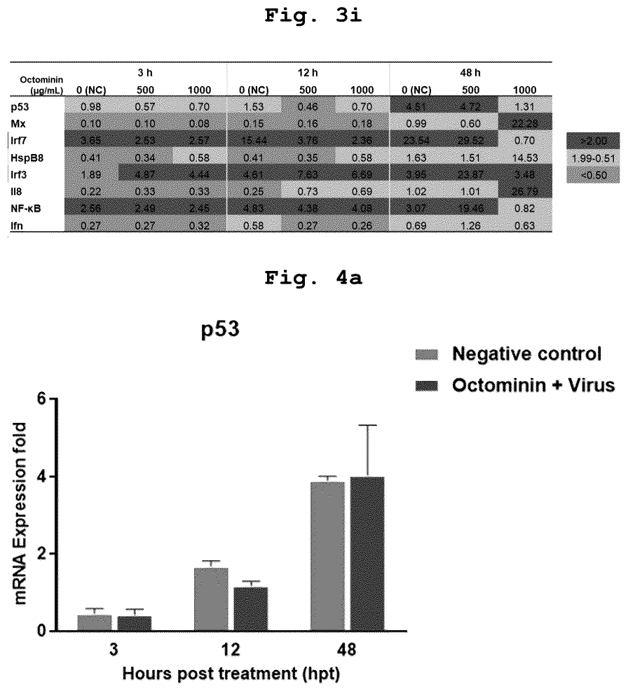

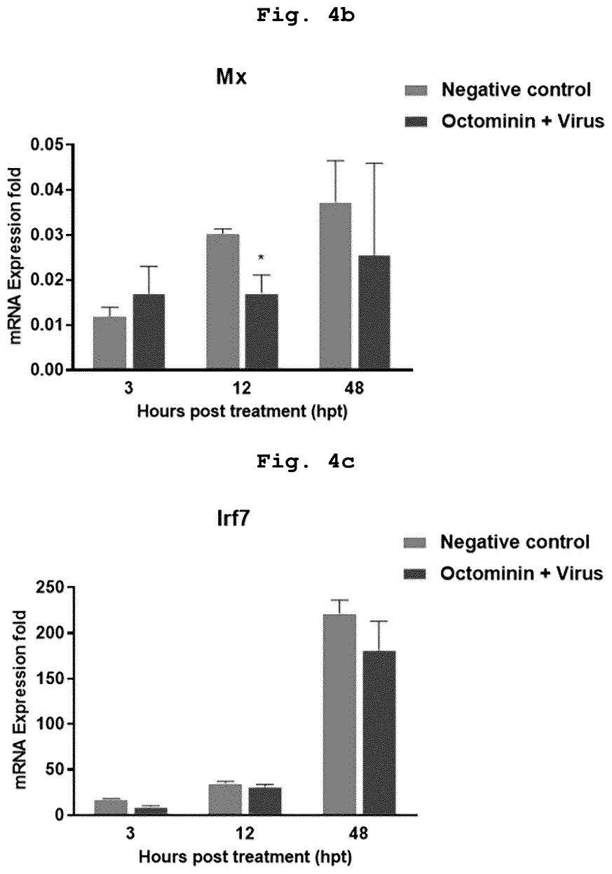

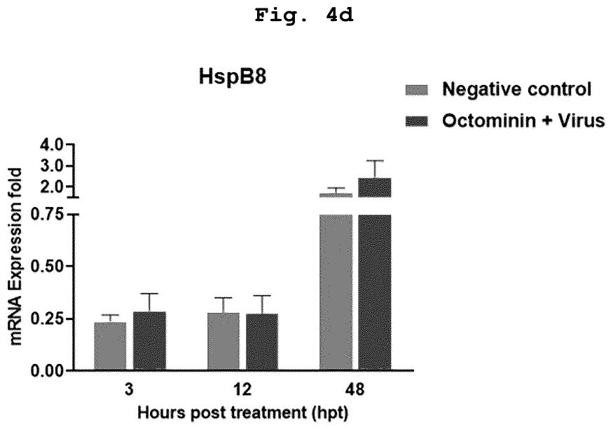

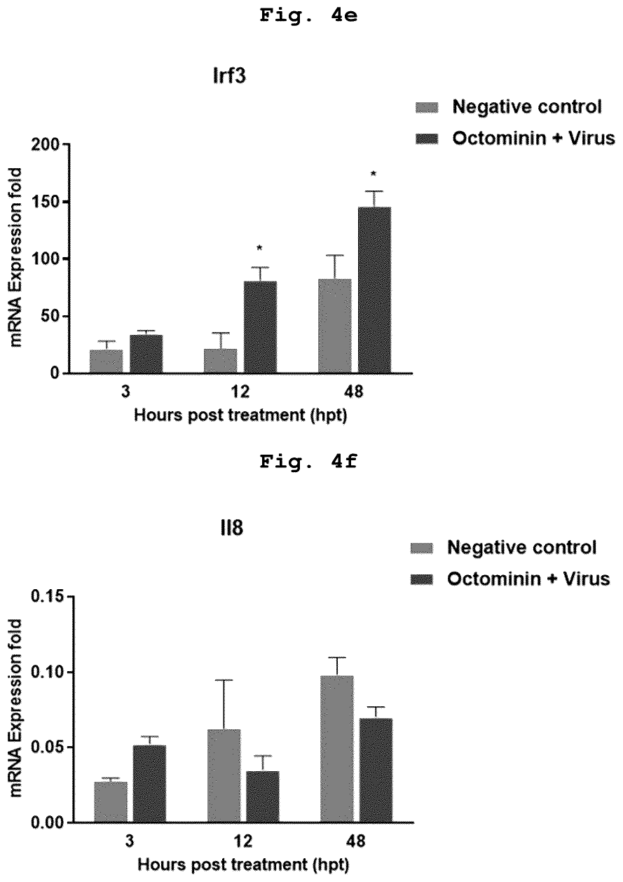

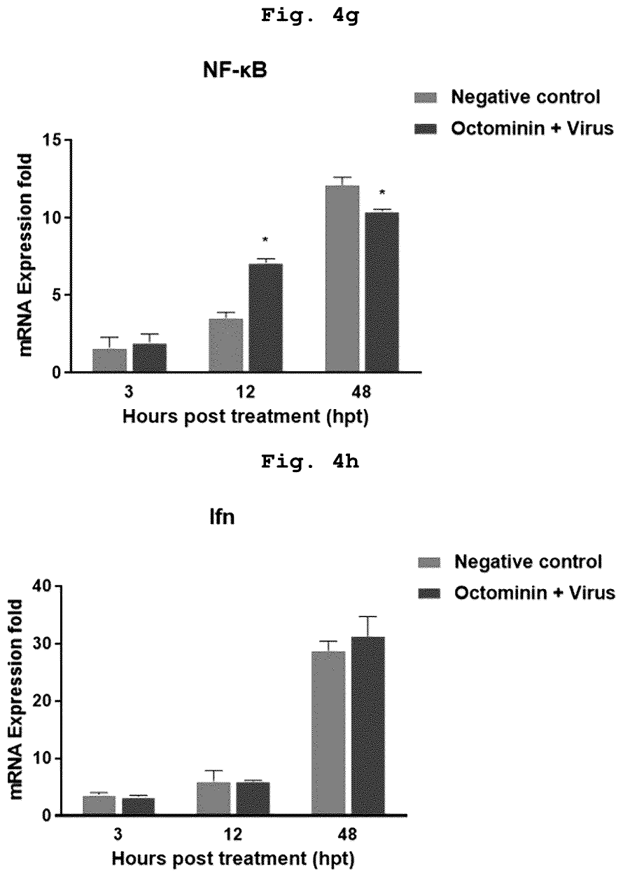

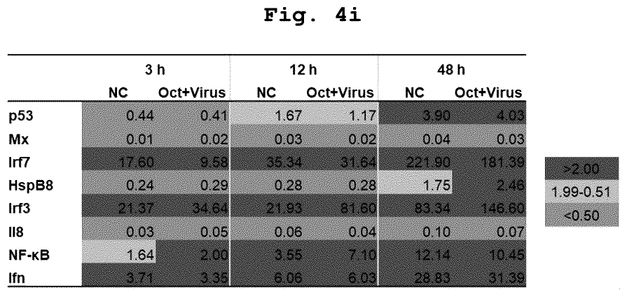

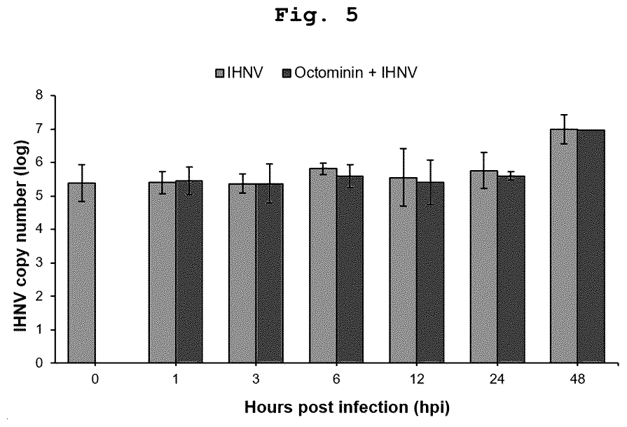

To confirm the antiviral activity of the Octominin peptide, the present inventors treated cells with Octominin and confirmed a cytopathic effect of three major fish pathogenic viruses VHSV (F1Wa05 strain), IHNV (RtPy91 strain), and IPNV (VR299 strain) (FIG. 1). As a result, it was confirmed that cytopathic effects occurred in all viruses, and studies were further conducted on IHNV having the highest activity. To investigate an antiviral activity mechanism, as a result of performing a plaque-forming reduction assay of Octominin on FHM cells infected with IHNV, it was confirmed that plaque formation was significantly reduced when Octominin was treated immediately before (−4 hpi) or immediately after (+1 hpi, +5 hpi) virus infection (see FIG. 2). In addition, it was confirmed that the expression levels of transcription factors such as Irf3, Irf7, and NF-KB, HspB8, and apoptosis function genes increased in cells treated with Octominin (see FIGS. 3A, 3B, 4A, and 4B). In addition, it was confirmed that the IHNV virus copy number was reduced by Octominin treatment (500 μg/mL) in FHM cells (see FIG. 5). In the present disclosure, at least three independent experiments were performed, and the data were expressed as the mean ±standard deviation (SD). Statistical analysis was performed using GraphPad Prism software version 8 (San Diego, CA, USA), and analysis of differences between control and treatment groups was performed by applying an unpaired t-test. The significance of the analyzed data was determined as p<0.05.

Hereinafter, the present disclosure will be described in more detail through Examples and Experimental Examples.

These Examples and Experimental Examples are just illustrative of the present disclosure, and it will be apparent to those skilled in the art that it is not interpreted that the scope of the present disclosure is limited to these Examples and Experimental Examples.

### <Example 1> Synthesis and Purification of Octominin

An Octominin peptide (GWLIRGAIHAGKAIHGLIHRRRH, SEQ ID NO: 1) was synthesized using a solid-phase peptide synthesis technique (AnyGen Co., Korea) and purified by reverse-phase HPLC using a SHIMADZU C18 analytical column (Shimadzu HPLC LabSolution, Japan).

### <Experimental Example 1> Confirmation of Antiviral and Cytopathic Effects of Octominin

To confirm the antiviral activity of Octominin obtained in Example 1, a cytopathic effect (CPE) reduction assay was performed.

Specifically, three major fish pathogenic viruses VHSV (F1Wa05 strain), IHNV (RtPy91 strain), and IPNV (VR299 strain) were used to study the antiviral activity of Octominin. Octominin at various concentrations reacted with an equal amount of virus (infectious titer: 200 TCID₅₀/mL) at 15° C. for 1 hour. Thereafter, the reactant was overlaid onto quadruplicate FHM cells in a 96-well plate (1×10⁵ cells/mL) and incubated for 96 hours. The observed cells were illustrated in FIG. 1.

CPE was scored by optical microscopy using a DIAPHOT 300 inverted microscope (Nikon Cooperation, Tokyo, Japan) (score 0, 0% CPE; score 1, 10 to 20% CPE; score 2, 30 to 40% CPE; score 3, 50 to 60% CPE; score 4, 70 to 80% CPE; and score 5, 90 to 100%). The results were expressed as a 50% effective concentration (EC₅₀) and calculated using Microsoft Excel software. Ribavirin, a known broad-spectrum antiviral agent, was used as a positive control group. The antiviral activity of Octominin was evaluated by calculation using Equation SI (selective index)=CC₅₀/EC₅₀.

TABLE 1

| Sample | Cell Line | Virus | CC₅₀ | EC₅₀ | SI |
|---|---|---|---|---|---|
| Octominin | FHM | VHSV | 2146.2 | 732.8 | 2.9 |
| (μg/mL) | | | (1139.6 μM) | (389.1 μM) | |
| | | IHNV | | 435.1 | 4.9 |
| | | | | (231.0 μM) | |
| | CHSE-214 | IPNV | 1865.2 | 925.9 | 2.0 |
| | | | (990.4 μM) | (491.7 μM) | |
| Ribavirin | FHM | VHSV | 962.5 | 0.27 | 3565.0 |
| (μM) | | IHNV | | 0.55 | 1750.1 |
| | CHSE-214 | IPNV | 121.4 | 0.75 | 161.9 |

As a result, as shown in Table 3, the CC₅₀ values for Octominin in FHM and CHSE-214 cells were 2146.2 μg/mL (1139.6 μM) and 1865.2 μg/mL (990.4 μM), respectively. The CC₅₀ values of ribavirin, used as a positive control group, were 962.5 μM and 121.4 μM in FHM and CHSE-214 cells, respectively.

In addition, the EC₅₀ values in Octominin-treated FHM cells were 732.8 μg/mL (389.1 μM) and 435.1 μg/mL (231.0 μM) in VHSV and IHNV challenges, respectively, whereas IPNV-challenged CHSE-214 cells showed an EC₅₀ value of 925.9 μg/mL (491.7 μM). However, compared to Octominin, ribavirin had the lowest EC₅₀ values in all cases (Table 3).

In addition, when FHM cells were treated with Octominin, it was confirmed that the SI (Selective index) value for IHNV was 4.9 to exhibit the highest antiviral activity compared to IPNV (SI 2.0) and VHSV (SI 2.9).

According to these results, a time course study of Octominin against IHNV was performed because Octominin was more active against IHNV than other viruses.

### <Experimental Example 2> Confirmation of Antiviral Activity Mechanism of Octominin

To investigate an antiviral activity mechanism of Octominin in FHM cells infected with IHNV, a plaque-forming reduction assay was performed.

Specifically, as shown in FIG. 2A, the following six treatment methods were followed based on time points −4, −1, 0, +1, +5 hpi, and +24 hpi at which cells were treated with Octominin.

### i) Pre-Treatment, −4 hpi

FHM cells (10⁶ cells/well) were pre-treated with Octominin (500 μg/mL, EC₅₀ standard, 435.1 μg/mL) for 4 hours at 15° C., washed with PBS, and inoculated with 200 μL of an IHNV suspension containing approximately 80 to 100 pfu at 15° C. for 1 hour, and then the cells were washed twice with PBS, and each well was overlaid with 1 mL of a methylcellulose medium (DMEM) containing 0.8% methylcellulose/2% FBS. After incubated at 15° C. for 7 days, infected cells were fixed with 10% formalin and stained with crystal violet. Then, the number of plaques was counted. Control cells were not treated with any sample.

### ii) External Incubation, −1 hpi

To investigate whether Octominin had a direct virus-killing effect, IHNV was pre-incubated with an equal amount of Octominin (500 μg/mL) at 15° C. for 1 h, and then the mixture was inoculated onto FHM cells (10⁶ cells/well). After viral adsorption, the cells were washed twice with PBS and each well was overlaid with 1 mL of a methylcellulose medium. The plate was incubated at 15° C. for 7 days and the number of plaques was counted.

iii) Co-Treatment, 0 hpi

FHM cells (10⁶ cells/well) were inoculated with equal volumes of Octominin (500 μg/mL) and IHNV at 15° C. for 1 hour, and then washed twice with PBS. Thereafter, each well was overlaid with the methylcellulose medium. The plate was incubated at 15° C. for 7 days and the number of plaques was counted.

### iv) Post-Treatment +1, +5, +24 hpi

FHM cells (10⁶ cells/well) were inoculated with virus at 15° C. for 1 hour and washed twice with PBS. Then, the cells were overlaid with the methylcellulose medium containing Octominin (500 μg/mL) immediately after 1, 5, and 24 hours. The plate was incubated at 15° C. for 7 days and the number of plaques was counted.

As a result, as shown in FIG. 2B, when the cells were pre-treated with Octominin (500 μg/mL) 4 hours (−4 hpi) before virus inoculation, the plaques were significantly reduced by 62.4±5.5% (P<0.001) compared to an IHNV alone group. In addition, it was confirmed that when FHM cells were exposed to IHNV for 1 hour (+1 hpi) and then treated with Octominin, the plaque formation was significantly reduced (35.9±3.6%, P<0.001), and when Octominin was treated 5 hours after virus exposure (+5 hpi), the plaque formation was slightly reduced (15.2±5.3%). However, when the cells were exposed to IHNV for 1 hour and Octominin was added after 24 hours (+24 hpi), there was no plaque reduction effect. Moreover, pre-incubation (-1 hpi) and co-incubation of the virus did not reduce the plaque formation compared to the IHNV control group. It was indicated that Octominin did not directly kill the virus or block the invasion of the virus.

### <Experimental Example 3> Confirmation of Changes in Gene Expression Levels by Octominin

### <3-1> Confirmation of Gene Expression Levels According to Octominin Treatment Concentration and Time

To understand changes in gene expression levels by Octominin, a quantitative real-time polymerase chain reaction (qRT-PCR) was performed to analyze the expression of immune-regulatory genes at various time points of 3, 12, and 48 hours after treatment with Octominin at various concentrations of 500 to 1000 μg/mL.

Specifically, the cells were treated with Octominin (500 to 1000 μg/mL) and incubated at 15° C. for 3, 12, and 48 hours. The cells were centrifuged at 1500×g for 10 minutes and RNA was isolated according to the manufacturer's protocol (NucleoSpin® RNA Kit, Macherey-Nagel, Duren, Germany). cDNA was synthesized using a Prime script 1st strand cDNA synthesis Kit (TaKaRa, Japan) according to the manufacturer's guidelines. qRT-PCR was performed by loading the total reaction mixture (10 μL) containing 5 μL of TB green Primix Ex Taq II, 3 μL of cDNA, and 1 μL (10 μM) each of forward and reverse primers into the Thermal Cycler Dice Real Time System III (TaKaRa, Japan). The primers used in the present disclosure were shown in Table 2 below. Fold values of relative mRNA expression were determined using a 2^{-(ΔΔCT)} method.

TABLE 2

| Gene | Primer | Sequence (5′-3) |
|---|---|---|
| IFN regulatory | Irf3-F | AGCATGCTTTGAGACAGGAC (SEQ ID NO: 2) |
| factor 3 (Irf3) | Irf3-R | CACGAAGAGOGCTACGGAAGTT (SEQ ID NO: 3) |
| IFN regulatory | Irf7-F | GTTCGTCTCAAAGTTGCTCCTC (SEQ ID NO: 4) |
| factor 7 (Irf7) | Irf7-R | GTTCGTCTCAAAGTTGCTCCTC (SEQ ID NO: 5) |
| Interferon (Inf) | Inf-F | CAACAACATCATGACCCGCTACCT (SEQ ID NO: 6) |
| | Inf-R | GTTCTCTGCCTCCGTTCTGTCCTT (SEQ ID NO: 7) |
| NF-KB | Nf-KB-F | GGAGAGGAGGTCTATCTGCTATG (SEQ ID NO: 8) |
| | Nf-KB-R | GGCTTCTGGAGGTTCTGGTC (SEQ ID NO: 9) |
| Mx1 | Mx-F | TGGCATGGGAGAATCAGTTACAAG (SEQ ID NO: 10) |
| | Mx-R | TGCCCCAGCCATCTCATCC (SEQ ID NO: 11) |
| Interleukin 8 (I18) | I18-F | CCCTCCTAGCCCTCACTGTAAA (SEQ ID NO: 12) |
| | I18-R | GGATCTTCTCAATGACCTTCTT (SEQ ID NO: 13) |
| tumor protein (p53) | p53-F | ACAGCAGTTGCATGGGTG (SEQ ID NO: 14) |
| | p53-R | TGGTCTCCTGGTCTTTCC (SEQ ID NO: 15) |
| Heat shock protein 8B | HspB8-F | CGAGCAGTACGCGTGGGAGTC (SBQ ID NO: 16) |
| (HspB8) | HspB8-R | AGCGTGATGGGGTAGCCGATGAAC (SEQ ID NO: 17) |
| beta-actin | beta-actin-F | CCGTGCTGTCTGGAGGTA (SEQ ID NO: 18) |
| | beta-actin-R | AAGGAGCAAGGGAGGTGATTTC (SEQ ID NO: 19) |
| IHNV- N gene | IHNV-NC-F | ATGACAAGCGCACTCAGAGAGA (SEQ ID NO: 20) |
| (copy number | IHNV-NC-R | TCAGTGGAATGAGTCGGAGTCTC (SEQ ID NO: 21) |
| analysis) | | |
| IHNV- N gene | IHNV-N-F | GCTTGCAGAAACGATCGTAAAG (SEQ ID NO: 22) |
| (qRT-PCR analysis) | IHNV-N-R | GGCCATCTTGTCCACATCATA (SEQ ID NO: 23) |

As a result, as shown in FIGS. 3A to 3I, only Irf7, Irf3, and NF-κB were up-regulated at 3 hpt. In addition, the expression of p53, IFN, and NF-κB was significantly increased at 48 hpt when the cells were treated with 500 μg/mL of Octominin, whereas no change was observed at 12 hpt. The expression levels of Mx and HSP had no large difference depending on a concentration of Octominin at 12 hpt, but were significantly up-regulated at 48 hpt in the treatment of 1000 μg/mL of Octominin compared to the treatment of 500 μg/mL of Octominin. The expression of Irf3 and IL was up-regulated at both concentrations at 12 hpt, whereas at 48 hpt, Irf3 was significantly up-regulated only at 500 μg/mL and IL8 was significantly up-regulated only at 1000 μg/mL. The expression of IFN7 was up-regulated in a 500 μg/mL treatment group at 48 hpt, but was significantly down-regulated in other cases.

### <3-2> Confirmation of Gene Expression Levels According to IHNV Infection and Octominin

Gene expression levels were confirmed after Octominin treatment 4 hours before IHNV challenge.

Specifically, before cell treatment, Octominin (500 μg/mL) was mixed with an equal amount of IHNV and reacted for 1 hour (infectious titer: 200 TCID₅₀/mL). Then, Octominin was transferred to 1×10⁶ cells/mL of FHM cells incubated at 15° C. for 24 hours and centrifuged. Thereafter, qRT-PCR analysis of other gene expression was performed in the same manner as in <3-1> above.

As a result, as shown in FIGS. 4A to 4I, compared to the control group, FHM cells treated with Octominin showed up-regulation of Irf3 at 12 hpt and 48 hpt, but NF-KB was up-regulated only at 12 hpt. It was confirmed that the gene expression of IFN, P53, and HsB8 was up-regulated only at 48 hpt compared to the control group. It was confirmed that the expression of IL8, Mx, and Irf7 was down-regulated at both 12 hpt and 48 hpt compared to the control group.

### <Experimental Example 4> Absolute Quantification of IHNV Copy Number in FHM Cells

qRT-PCR was performed to confirm changes in the copy number of nucleocapsid-N gene of IHNV by Octominin.

Specifically, the N gene was selected due to association with viral virulence. IHNV RNA was isolated using the NucleoSpin® RNA Virus kit (Macherey-Nagel, Duren, Germany). A PCR product was amplified using primers that amplified the N gene of IHNV (see Table 2) and confirmed by agarose gel electrophoresis. Next, the amplified PCR product was ligated into a pGEM®-T Easy Vector (Promega, Madison, WI, USA) and sequenced to confirm the N gene. Absolute quantification of the N gene expression was performed and a standard curve was plotted with a transformed plasmid concentration and a threshold cycle (Ct). Meanwhile, seeded FHM cells were treated with 500 μg/mL of Octominin and IHNV challenge was performed after 1 hour. The FHM cells were harvested at 0, 1, 3, 6, 12, 24, and 48 hours (hpi) after challenge and subjected to qRT-PCR. Ct values were used together with a standard curve to obtain a distribution of virus copy numbers over time.

As a result, as shown in FIG. 5, it was confirmed that when treated with Octominin, virus replication was reduced in a time-dependent manner after 6 hpi compared to a cell group challenged only with IHNV virus.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An antiviral composition for fish viral diseases comprising a peptide having an amino acid sequence as set forth in SEQ ID NO: 1 as an active ingredient.

2. The antiviral composition for fish viral diseases of claim 1, wherein the fish viral diseases are diseases caused by at least one virus infection selected from the group consisting of viral hemorrhagic septicemia virus (VHSV), infectious hematopoietic necrosis virus (IHNV), and infectious pancreatic necrosis virus (IPNV).

3. The antiviral composition for fish viral diseases of claim 1, wherein the fish is at least one selected from the group consisting of halibut, rockfish, red sea bream, rock bream, sea bass, gray mullet, bass, Gizzard shad, flounder, puffer fish, mackerel, greenling, tuna, croaker, yellowtail, horse mackerel, salmon, rainbow trout, carp, leather carp, eel, catfish, loach, and crucian carp.

4. A composition for a fish feed additive having antiviral activity against fish viral diseases comprising a peptide having an amino acid sequence as set forth in SEQ ID NO: 1 as an active ingredient.

5. The composition for a fish feed additive of claim 4, wherein the fish viral diseases are diseases caused by at least one virus infection selected from the group consisting of viral hemorrhagic septicemia virus (VHSV), infectious hematopoietic necrosis virus (IHNV), and infectious pancreatic necrosis virus (IPNV).

6. A method for preventing or treating a fish viral disease, the method comprising administering to a fish a peptide having an amino acid sequence as set forth in SEQ ID NO: 1.

7. The composition for a fish feed additive of claim 4, wherein the fish is at least one selected from the group consisting of halibut, rockfish, red sea bream, rock bream, sea bass, gray mullet, bass, Gizzard shad, flounder, puffer fish, mackerel, greenling, tuna, croaker, yellowtail, horse mackerel, salmon, rainbow trout, carp, leather carp, eel, catfish, loach, and crucian carp.

8. The method of claim 7, wherein the fish viral diseases are diseases caused by at least one virus infection selected from the group consisting of viral hemorrhagic septicemia virus (VHSV), infectious hematopoietic necrosis virus (IHNV), and infectious pancreatic necrosis virus (IPNV).

9. The method of claim 7, wherein the fish is at least one selected from the group consisting of halibut, rockfish, red sea bream, rock bream, sea bass, gray mullet, bass, Gizzard shad, flounder, puffer fish, mackerel, greenling, tuna, croaker, yellowtail, horse mackerel, salmon, rainbow trout, carp, leather carp, eel, catfish, loach, and crucian carp.
